(19) United States
(12) Patent Application Publication
(10) Pub. No.: US 2015/0165380 A1
(43) Pub. Date: Jun. 18, 2015

(54) DRAW SOLUTES COMPRISING ALKYL AMMONIUM SALT COMPOUNDS

(71) Applicant: Samsung Electronics Co., Ltd., Suwon-Si (KR)

(72) Inventors: Won Cheol JUNG, Seoul (KR); Seung Rim YANG, Seongnam-si (KR); Chang Won SUH, Incheon-si (KR); Bo Kyung JUNG, Yongin-si (KR); Sung Soo HAN, Hwaseong-si (KR)

(21) Appl. No.: 14/565,121

(22) Filed: Dec. 9, 2014

(30) Foreign Application Priority Data
Dec. 12, 2013 (KR) 10-2013-0154944

(51) Int. Cl. B01D 61/00; C07C 211/14; C07C 211/09; C02F 1/44; C08G 73/02

(52) U.S. Cl. CPC B01D 61/005; C02F 1/445; C08G 73/0206; C07C 211/09; C07C 211/14; C02F 2103/08

(57) ABSTRACT

A draw solute includes a water-soluble alkyl ammonium salt compound, the water-soluble alkyl ammonium salt compound including an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation.

### CROSS-REFERENCE TO RELATED APPLICATION

This application claims priority under 35 U.S.C. §119 to Korean Patent Application No. 10-2013-0154944, filed in the Korean Intellectual Property Office on Dec. 12, 2013, the entire contents of which are incorporated herein by reference.

### BACKGROUND

1. Field

Example embodiments relate to draw solutes including alkyl ammonium salt compounds, forward osmosis water treatment devices and methods using the same.

2. Description of the Related Art

Osmosis (or forward osmosis) refers to a phenomenon wherein water moves from a lower solute concentration solution to a solution of a higher solute concentration by osmotic pressure. Reverse osmosis is a method of artificially applying pressure to move water in the opposite direction.

Desalination through reverse osmosis is a known technique in the field of water treatment. Reverse osmosis desalination involves artificially applying a relatively high pressure and thus requires relatively high energy consumption. To increase energy efficiency, a forward osmosis process using the principle of osmotic pressure has been suggested, and as a solute for the osmosis draw solution, ammonium bicarbonate, sulfur dioxide, aliphatic alcohols, aluminum sulfate, glucose, fructose and potassium nitrate have been used. Among them, an ammonium bicarbonate draw solution is most commonly used, and after the forward osmosis process, the draw solute (e.g., ammonium bicarbonate) may undergo decomposition into ammonia and carbon dioxide at a temperature of about 60° C. and be removed. Further, newly suggested draw solutes include magnetic nanoparticles having hydrophilic polymers (e.g., peptides and low molecular weight materials attached thereto (that can be separated by a magnetic field)) and/or a polymer electrolyte (e.g., a dendrimer (that can be separated by an ultrafiltration (UF) or nanofiltration (NF) membrane)).

Because decomposition of ammonium bicarbonate requires heating at about 60° C. or higher, removal of the draw solute including the above compound requires a relatively high level of energy consumption. In addition, because complete elimination of ammonia is difficult (if not impossible), water produced by forward osmosis using ammonium bicarbonate as the draw solute is typically not suitable for drinking water due to the odor of ammonia. Meanwhile, magnetic nanoparticles present difficulties in terms of redispersing the agglomerated particles being separated from the draw solution by using a magnetic field. Completely removing the nanoparticles may also be difficult (if not impossible). Thus, the toxicity of the nanoparticles may be an additional disadvantage. Polyionic draw solutes may generate a relatively high level of osmotic pressure, but they tend to diffuse into a feed solution, which leads to severe loss of the draw solute. In addition, the recovery of the draw solutes requires a tight nano-filtration membrane and thus requires a relatively high energy process. Moreover, most of the draw solutes generally exhibit a relatively high level of toxicity, and therefore are typically difficult to use in a forward osmosis process for producing drinking water.

### SUMMARY

Some example embodiments relate to alkyl ammonium salt based draw solutes that may realize relatively high water flux and relatively low reverse solute flux, and may exhibit a relatively low level of toxicity.

Some example embodiments relate to forward osmosis water treatment devices and methods using a draw solution including such draw solutes.

According to example embodiments, a draw solute includes a water-soluble alkyl ammonium salt compound including an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation.

The ionic moiety may include one of —COOM, —SO₃M, —OSO₃M, —OPO₃M₂, —OPO₃MH, —PO₃M₂, —OPO₃Me and a combination thereof, wherein M is one of Li, Na, K and Rb, and Me is one of Ca, Mg, Sr and Ba.

The at least two ammonium cationic moieties may include one of a primary ammonium cation, a secondary ammonium cation, a tertiary ammonium cation and a combination thereof.

The water-soluble alkyl ammonium salt compound may include a reaction product between a polyamine compound having at least two amine groups and one of an organic acid salt having the ionic moiety together with a functional group capable of donating hydrogen to one of the at least two amine groups and an acid salt having the ionic moiety.

The at least two amine groups may be the same or different and may be independently selected from one of a primary amine, a secondary amine, and a tertiary amine, and the functional group capable of donating hydrogen to the at least two amine groups may be one of a carboxyl group (—COOH), a sulfonic acid group (—SO₃H), a phosphonic acid group (—PO₃H₂), a phosphoric acid group (—OPO₃H₂) and a combination thereof.

The polyamine compound may be a compound represented by Chemical Formula 1:

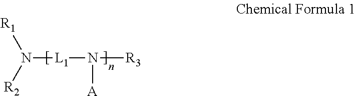

wherein R₁, R₂, and R₃ are the same or different, and are each independently one of a hydrogen and a substituted or unsubstituted C₁ to C₃₀ monovalent aliphatic hydrocarbon group, L₁ is a substituted or unsubstituted C₁ to C₃₀ divalent aliphatic hydrocarbon group, A is one of a hydrogen, a substituted or unsubstituted C₁ to C₃₀ monovalent aliphatic hydrocarbon group, a substituted or unsubstituted aminoalkyl group, and a moiety represented by Chemical Formula 2, n is an integer of greater than or equal to 1, and when n is at least 2, each L₁ is the same or different and each A is the same or different:

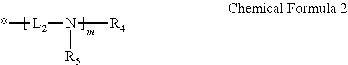

wherein L₂ is a substituted or unsubstituted C₁ to C₃₀ divalent aliphatic hydrocarbon group, R₄ and R₅ are the same or different and are each independently one of a hydrogen, a substituted or unsubstituted C₁ to C₃₀ monovalent aliphatic hydrocarbon group, and a moiety represented by Chemical Formula 2, m is an integer greater than or equal to 1, and * is a portion that is linked to the nitrogen atom.

The polyamine compound may include one of polyethylene imine, polypropylene imine, bishexamethylenetriamine, ethylenediamine, 1,2-diamino propane, 1,3-diamino propane, N-methylene diamine, 1,4-diamino butane, 3-(methylamino)propylamine, N,N′-dimethylethylenediamine, N,N-dimethylethylenediamine, N-ethylethylenediamine, N-methyl-1,3-diamino propane, 1-dimethyl amino2-propylamine, 3-(dimethyl amino)-1-propylamine, Cadaverine, N,N′-dimethyl-1,3-propanediamine, N,N,N′,N′-tetramethyldiaminomethane, N,N,N′-trimethylethylenediamine, N-isopropylethylenediamine, N-propylethylenediamine, 2-(aminomethyl)-2-methyl-1,3-propanediamine, 2-(aminomethyl)-2-methyl-1,3-propanediamine trichloride, N-(2-aminoethyl)-1,3-propanediamine, 1,5-diamino-2-methylpentane, N,N′-diethylethylenediamine, N,N,N′,N′-tetramethylethylenediamine, N,N-diethylethylenediamine, N,N-dimethyl-N′-ethylethylenediamine, N-butylethylenediamine, N-isopropyl-1,3-propanediamine, N-propyl-1,3-propanediamine, bis(3-aminopropyl)amine, triethylenetetramine, 1,3-bis(ethylamino)propane, 1,7-diamino heptane, 3-(diethylamino)propylamine, N,N′-diethyl-1,3-propanediamine, N,N,2,2-tetramethyl-1,3-propanediamine, N,N,N′,N′-Tetramethyl-1,3-propanediamine, N,N-diethyl-1,3-propanediamine, N,N-diethyl-N′-methylethylenediamine, 3,3′-diamino-N-methyldipropylamine, N′-isopropyldiethylenetriamine), tris(dimethylamino)methane, N,N′-bis(2-aminoethyl)-1,3-propanediamine, trans-N,N,N′,N′-tetramethyl-2-butene-1,4-diamine, 1,8-diaminooctane, N,N′-dimethyl-1,6-hexane diamine, N,N,N′,N′-tetramethyl-1,3-butanediamine, N,N,N′-triethylethylenediamine, N-hexyl ethylenediamine, bis[2-(N,N-dimethyl amino)ethyl]ether, N,N-diethyldiethylenetriamine, N,N-dimethyl dipropylene triimine, 1,2-bis(3-aminopropylamino)ethane, tetraethylene pentamine, 2,4,6-trimethyl-m-phenylenediamine, N-benzylethylenediamine, and a combination thereof.

The organic acid salt may include an alkali metal salt of a polycarboxylic acid, and the acid salt may include one of sodium bisulfate, monosodium phosphate, disodium phosphate and a combination thereof.

The alkali metal salt of a polycarboxylic acid may include one of an alkali metal salt of a C₂ to C₄₀ dicarboxylic acid (e.g., a monoalkali metal salt), an alkali metal salt of a C₃ to C₄₀ tricarboxylic acid (e.g., a mono alkali metal salt or a dialkali metal salt), an alkali metal salt of a C₄ to C₄₀ tetracarboxylic acid (e.g., a mono alkali metal salt, a dialkali metal salt, or a trialkali metal salt), an alkali metal salt of a C₅ to C₄₀ pentacarboxylic acid (e.g., a mono alkali metal salt, a dialkali metal salt, a trialkali metal salt, or a tetra alkali metal salt), an alkali metal salt of a C₆ to C₄₀ hexacarboxylic acid (e.g., a mono alkali metal salt, a dialkali metal salt, a trialkali metal salt, a tetra alkali metal salt, or a penta alkali metal salt), an alkali metal salt of C₄ to C₄₀ phosphonotricarboxylic acid and a combination thereof.

The water-soluble alkyl ammonium compound may have a molecular weight of greater than or equal to about 200 g/mol.

The water-soluble alkyl ammonium compound may include a polymeric compound having a number average molecular weight of greater than or equal to about 2,000 g/mol.

The draw solute may have a water flux of greater than or equal to about 10 LMH and a reverse solute flux of less than or equal to about 2 GMH at an osmotic pressure of about 60 atm.

According to example embodiments, a forward osmosis water treatment device includes a feed solution including water and materials dissolved in the water, an osmosis draw solution including water and a draw solute including the foregoing water-soluble alkyl ammonium salt compound, a semi-permeable membrane contacting the feed solution on a first side and the osmosis draw solution on an opposing second side, a recovery system configured to remove at least a portion of the draw solute from a treated solution including the water from the feed solution that moves to the osmosis draw solution through the semipermeable membrane by osmotic pressure, and a connector configured to reintroduce the draw solute removed by the recovery system back into the osmosis draw solution contacting the semi-permeable membrane.

The forward osmosis water treatment device may further include an outlet configured to discharge treated water produced by removing the draw solute from the treated solution in the recovery system.

The recovery system may include one of a microfiltration (MF) membrane, an ultrafiltration (UF) membrane, a loose nanofiltration (NF) membrane, a centrifugal separator, and a membrane distillation.

According to example embodiments, a forward osmosis method for water treatment may include contacting a feed solution, the feed solution including water and materials dissolved in the water, and an osmosis draw solution including a draw solute with a semi-permeable membrane therebetween to obtain a treated solution including the water that moves from the feed solution to the osmotic draw solution through the semi-permeable membrane by osmotic pressure, the draw solute including the aforementioned water-soluble alkyl ammonium salt compound, removing at least a portion of the draw solute from the treated solution to obtain treated water, and discharging the treated water.

The aforementioned alkyl ammonium salt compound may have a molecular structure designed relatively freely and may be more easily made to have a desired molecular weight. A draw solute including the alkyl ammonium salt compound may generate a relatively high level of osmotic pressure, may show a relatively low level of reverse solute flux, and may be more easily recovered after the water treatment. Therefore, the alkyl ammonium salt compound may find greater utility in the fields of water treatment, for example, using a forward osmotic pressure.

### BRIEF DESCRIPTION OF THE DRAWINGS

FIG. 1 is a schematic view of a forward osmosis water treatment device according to example embodiments.

FIGS. 2 to 5 are ¹H-NMR analysis spectrums of the alkyl ammonium salt compounds prepared in the synthesis examples.

FIG. 6 is a graph illustrating the changes of the water flux over the osmotic pressure in the experimental examples.

FIG. 7 is a graph illustrating the changes of the reverse solute flux over the osmotic pressure in the experimental examples.

### DETAILED DESCRIPTION

It will be understood that when an element or layer is referred to as being “on,” “connected to,” “coupled to,” or “covering” another element or layer, it may be directly on, connected to, coupled to, or covering the other element or layer, or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It will be understood that, although the terms first, second, third, etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the example embodiments.

Spatially relative terms, e.g., “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various embodiments only and is not intended to be limiting of the example embodiments. As used herein, the singular forms “a,” “an,” and “the” are intended to include the plural forms as well, unless the context clearly indicates otherwise. It will be further understood that the terms “comprises,” “comprising,” “includes,” and/or “including,” if used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art. It will be further understood that terms, including those defined in commonly used dictionaries, should be interpreted as having a meaning that is consistent with their meaning in the context of the relevant art and will not be interpreted in an idealized or overly formal sense unless expressly so defined herein.

As used herein, the term “substitute” refers to replacing one or more of hydrogen in a given group with a hydroxyl group, a nitro group, a cyano group, an amino group (—NR₂, R is hydrogen or C₁ to C₃₀ alkyl group), a carboxyl group, a linear or branched C₁ to C₃₀ alkyl group, a C₁ to C₁₀ alkyl silyl group, a C₃ to C₃₀ cycloalkyl group, a C₆ to C₃₀ aryl group, a C₂ to C₃₀ heteroaryl group, a C₁ to C₁₀ alkoxy group, a halogen, or a C₁ to C₁₀ fluoro alkyl group.

As used herein, the term “acid salt” refers to a salt that is formed by the partial neutralization of diprotic or polyprotic acids and has at least one exchangeable hydrogen atom.

In the example embodiments, a draw solute includes a water-soluble alkyl ammonium salt compound that includes an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation. The ionic moiety may include —COOM, —SO₃M, —OSO₃M, —OPO₃M₂, —PO₃M₂, —PO₃MH, —OPO₃Me, or a combination thereof, wherein M is Li, Na, K, or Rb, and Me is Ca, Mg, Sr, or Ba. In non-limiting examples, the ionic moiety may include —COONa, —SO₃Na, —OSO₃Na, —OPO₃Na₂, —PO₃Na₂, —PO₃NaH, —OPO₃Ca, or a combination thereof.

The alkyl ammonium salt compound includes an ionic moiety having an anion and its counter ion together with at least two ammonium cationic moieties. The alkyl ammonium salt compound may include at least two ammonium cationic moieties selected from a primary ammonium cation, a secondary ammonium cation, and a tertiary ammonium cation.

Due to having such a structure, the alkyl ammonium salt compound may show water solubility even when the alkyl ammonium salt compound is designed to have a relatively high molecular weight, may generate a relatively high level of osmotic pressure when the alkyl ammonium salt compound is dissolved in water, and may keep a rate of reverse solute flux at a relatively low level. Conventional draw solutes including a polyionic (e.g., polyvalent ionic) compound (e.g., MgCl₂, MgSO₄) may generate a relatively high level of osmotic pressure when they are dissolved in a large amount, but at the same time, they tend to show a relatively high level of reverse solute flux, which then leads to a relatively high loss and a relatively low recovery rate thereof, and also results in deterioration of the purity of the treated water. In particular, a “cake enhanced osmotic pressure” phenomenon may occur wherein the draw solute moved to the feed solution is confined in a fouling layer formed adjacent to the semipermeable membrane, causing an increase of osmotic pressure. Such a phenomenon may cause the osmotic pressure determined at the membrane surface contacting the feed solution to increase, resulting in a significant decrease in effective osmotic pressure and a lowered water flux.

If the osmotic pressure generated by the draw solute is to be effectively utilized in the forward osmosis water treatment, the draw solute should be allowed to readily pass through the porous layer of the semipermeable membrane and more easily reach the active layer, and at the same time, it should be difficult for the draw solute to pass through the dense active layer of the semipermeable membrane and move toward the feed solution. However, the draw solute (for example, including the low molecular weight compound) may more easily pass not only through the porous layer of the semipermeable membrane but also through the dense active layer thereof, and tends to move toward the feed solution (e.g., tends to cause reverse solute flux). On the other hand, when a relatively large molecular weight compound is used to control the moving of the solute toward the feed solution (e.g., the reverse solute flux), it is difficult to obtain a relatively high level of osmotic pressure, and the draw solute may have difficulties in passing through the porous layer of the semipermeable membrane to reach the active layer. Therefore, the effective osmotic pressure determined near the dense layer of the semipermeable membrane is lower than the originally obtainable osmotic pressure of the draw solute, and this may lead to a reduced water flux.

By contrast, the draw solute including the aforementioned alkyl ammonium salt compound may generate a relatively high level of effective osmotic pressure determined near the dense layer while keeping the reverse solute flux at a relatively low level (e.g., at a level of less than one third of the amount of the reverse solute flux caused by the polyionic compound (e.g., MgCl₂ and/or MgSO₄). Therefore, the draw solute including the aforementioned alkyl ammonium salt compound may avoid a decrease of the water flux over the water treatment time, may prevent or suppress the loss of the draw solute, and may show a relatively high draw solute recovery rate. In particular, even when the alkyl ammonium salt compound is designed to have a relatively high molecular weight (e.g., several tens of thousands, several hundreds of thousands, or several millions), the alkyl ammonium salt compound may be still dissolved in water and thus may generate a relatively high level of osmotic pressure and more easily pass the porous layer to reach the dense layer, thereby showing enhanced water flux. Moreover, the draw solute reaching the dense layer does not readily pass through the semipermeable membrane to move toward the feed solution, and thus the draw solute may keep the reverse solute flux at a reduced level. In addition, the recovery process may be accomplished via a relatively low energy process, e.g., by using a loose nano-filtration membrane.

The alkyl ammonium salt compound may be prepared by a relatively simple synthesis process. By way of an example, the alkyl ammonium salt compound may be a reaction product of a polyamine with an organic acid or an acid salt, wherein the polyamine has at least two amine groups, the organic acid salt has a functional group capable of donating hydrogen to the amine group and the aforementioned ionic moiety together, and the acid salt has the aforementioned ionic moiety. The reaction product may not only have an ammonium cation formed by the reaction between the amine group and the acid (or the functional group), but also include the ionic moiety derived from the organic acid salt or the acid salt. When the alkyl ammonium salt compound is treated with a strong base (e.g., sodium hydroxide and/or potassium hydroxide), the ammonium salt cation linkage of the reaction compound may be broken to produce the polyamine and the organic acid salt (or the acid salt) that are used as a raw material.

The polyamine compound may be a compound represented by Chemical Formula 1:

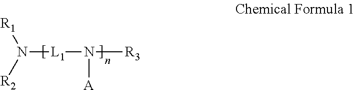

wherein R₁, R₂, and R₃ are the same or different, and are each independently hydrogen or a substituted or unsubstituted C₁ to C₃₀ monovalent aliphatic hydrocarbon group, L₁ is a substituted or unsubstituted C₁ to C₃₀ divalent aliphatic hydrocarbon group, A is hydrogen, a substituted or unsubstituted C₁ to C₃₀ monovalent aliphatic hydrocarbon group, a substituted or unsubstituted aminoalkyl group, or a moiety represented by Chemical Formula 2, n is an integer of greater than or equal to 1, and when n is at least 2, each of L₁ is the same or different and each of A is the same or different:

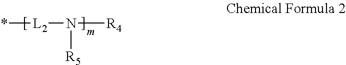

wherein L₂ is a substituted or unsubstituted C₁ to C₃₀ divalent aliphatic hydrocarbon group, R₄ and R₅ are the same or different and are each independently hydrogen, a substituted or unsubstituted C₁ to C₃₀ monovalent aliphatic hydrocarbon group (e.g., a substituted or unsubstituted C₁ to C₃₀ alkyl group, a substituted or unsubstituted C₆ to C₃₀ aryl group, and a substituted or unsubstituted C₆ to C₃₀ arylakyl group), or a moiety represented by Chemical Formula 2 (e.g., an aminoalkyl group), m is an integer greater than or equal to 1, and * is a portion that is linked to the nitrogen atom.

In example embodiments, in the Chemical Formula 1, R₁, R₂, and R₃ are the same as or different from each other, and may independently be hydrogen or a substituted or unsubstituted C₁ to C₃₀ alkyl group, L₁ may be a substituted or unsubstituted C₁ to C₃₀ alkylene group, a substituted or unsubstituted C₁ to C₃₀ alkenylene group, a substituted or unsubstituted C₆ to C₂₀ cycloalkylene group, or a substituted or unsubstituted C₆ to C₂₀ cycloalkenylene group, A may be hydrogen, a substituted or unsubstituted C₁ to C₃₀ alkyl group, a substituted or unsubstituted C₆ to C₃₀ aryl group, a substituted or unsubstituted C₆ to C₃₀ arylakyl group, a substituted or unsubstituted aminoalkyl group, or a moiety represented by the above Chemical Formula 2.

In example embodiments, in the Chemical Formula 2, L₂ may be a substituted or unsubstituted C₁ to C₃₀ alkylene group, a substituted or unsubstituted C₁ to C₃₀ alkenylene group, a substituted or unsubstituted C₆ to C₂₀ cycloalkylene group, or a substituted or unsubstituted C₆ to C₂₀ cycloalkenylene group, and R₄ and R₅ are the same or different from each other, and may independently be hydrogen, a substituted or unsubstituted C₁ to C₃₀ alkyl group, a substituted or unsubstituted C₆ to C₃₀ aryl group, a substituted or unsubstituted C₆ to C₃₀ arylakyl group, or a substituted or unsubstituted aminoalkyl group.

Examples of the polyamine compound may include, but are not limited to polyethylene imine, polypropylene imine, bishexamethylenetriamine, ethylenediamine, 1,2-diamino propane, 1,3-diamino propane, N-methylene diamine(N-methylethylenediamine), 1,4-diamino butane, 3-(methyl amino)propylamine (3-(methylamino)propylamine), N,N′-dimethyl ethylenediamine(N,N′-dimethylethylenediamine), N,N-dimethyl ethylenediamine(N,N-dimethylethylenediamine), N-ethylethylenediamine, N-methyl-1,3-diamino propane, 1-dimethyl amino2-propylamine, 3-(dimethyl amino)-1-propylamine, Cadaverine, N,N′-dimethyl-1,3-propanediamine, N,N,N′,N′-tetramethyldiaminomethane, N,N,N′-trimethylethylenediamine, N-isopropylethylenediamine, N-propylethylenediamine, 2-(aminomethyl)-2-methyl-1,3-propanediamine, 2-(aminomethyl)-2-methyl-1,3-propanediamine trichloride, N-(2-aminoethyl)-1,3-propanediamine, 1,5-diamino-2-methylpentane, N,N′-diethylethylenediamine, N,N,N′,N′-tetramethyl ethylenediamine, N,N-diethylethylenediamine, N,N-dimethyl-N′-ethylethylenediamine, N-butylethylenediamine, N-isopropyl-1,3-propanediamine, N-propyl-1,3-propanediamine, bis(3-aminopropyl)amine, triethylenetetramine, 1,3-bis(ethylamino)propane, 1,7-diamino heptane, 3-(diethylamino)propylamine, N,N′-diethyl-1,3-propanediamine, N,N,2,2-tetramethyl-1,3-propanediamine, N,N,N′,N′-tetramethyl-1,3-propanediamine, N,N-diethyl-1,3-propanediamine, N,N-diethyl-N′-methylethylenediamine, 3,3′-diamino-N-methyl dipropylamine, N′-isopropyl diethylenetriamine, tris(dimethylamino)methane, N,N′-bis(2-aminoethyl)-1,3-propanediamine, trans-N,N,N′,N′-tetramethyl-2-butene-1,4-diamine, 1,8-diamino octane, N,N′-dimethyl-1,6-hexane diamine, N,N,N′,N′-tetramethyl-1,3-butanediamine, N,N,N′-triethylethylenediamine, N-hexyl ethylenediamine, bis[2-(N,N-dimethylamino)ethyl]ether, N,N-diethyldiethylenetriamine, N,N-dimethyldipropylene triimine, 1,2-bis(3-aminopropylamino)ethane, or tetraethylene pentamine.

The polyamine may be a polyamine polymer (e.g., polyethylene imine and polypropylene imine). The molecular weight of the polyamine polymer is not particularly limited, but may be selected appropriately. For example, when being measured with gel permeation chromatography, the number average molecular weight of the polyamine polymer may be, for example, greater than or equal to about 600 g/mol, greater than or equal to about 1000 g/mol, greater than or equal to about 2000 g/mol, greater than or equal to about 3000 g/mol, greater than or equal to about 4000 g/mol, greater than or equal to about 5000 g/mol, or greater than or equal to about 60,000. The number average molecular weight of the polyamine polymer may be less than or equal to about 1,000,000 g/mol, less than or equal to about 900,000 g/mol, less than or equal to about 800,000 g/mol, less than or equal to about 700,000 g/mol, less than or equal to about 600,000 g/mol, or less than or equal to about 500,000 g/mol.

For example, the polyethylene imine may be represented by Chemical Formula 3.

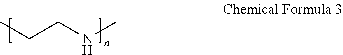

wherein the number “n” corresponds to a degree of polymerization of the polyethylene imine.

For example, the polyethylene imine may have a linear structure as illustrated below:

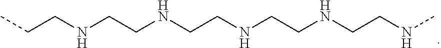

For example, the polyethylene imine may have a branched structure as illustrated below:

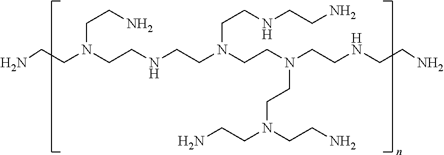

wherein the number “n” corresponds to a degree of polymerization of the polyethylene imine.

For example, the polyethylene imine may have a dendrimer structure as illustrated below:

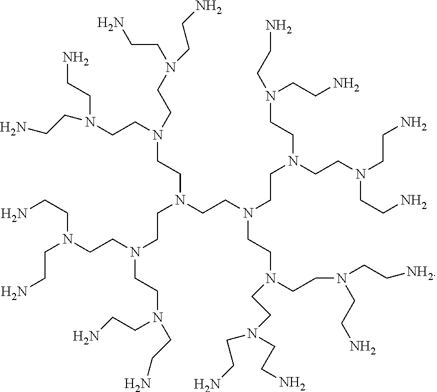 [SMILES: NCCN(CCN)CCN(CCN(CCN)CCN)CCN(CCN(CCN(CCN)CCN)CCN(CCN)CCN)CCN(CCN(CCN(CCN)CCN)CCN(CCN)CCN)CCN(CCN(CCN)CCN)CCN(CCN)CCN]

The aforementioned polyamine may be synthesized in any known method or is commercially available.

In the polyamine, the at least two amine groups are the same or different and are independently a primary amine, a secondary amine, or a tertiary amine. When the polyamine has a primary amine group, the alkyl ammonium salt compound thus prepared has a primary ammonium salt moiety. When the polyamine has a secondary amine group, the alkyl ammonium salt compound thus prepared has a secondary ammonium salt moiety. When the polyamine has a tertiary amine group, the alkyl ammonium salt compound thus prepared has a tertiary ammonium salt moiety.

The organic acid salt may have a functional group capable of donating hydrogen to the amine group together with the ionic moiety. The acid salt may have the ionic moiety. The functional group capable of donating hydrogen to the amine group may be a carboxyl group (—COOH), a sulfonic acid group (—SO₃H), a phosphonic acid group (—PO₃H₂), a phosphoric acid group (—OPO₃H₂), or a combination thereof. The organic acid salt may be an alkali metal salt of a polycarboxylic acid. The polycarboxylic acid may include a substituted or unsubstituted C₁ to C₄₀ divalent aliphatic hydrocarbon group, a substituted or unsubstituted C₆ to C₄₀ divalent aromatic hydrocarbon group, a substituted or unsubstituted C₃ to C₂₀ divalent aliphatic ether group, a substituted or unsubstituted C₃ to C₂₀ divalent cyclic ether group, a hydroxyl group, or a thioether group.

In non-limiting examples, the organic acid salt may be a mono-alkali metal salt of a C₂ to C₄₀ dicarboxylic acid (e.g., oxalic acid, malonic acid, succinic acid, glutaric acid, adipic acid, pimelic acid, suberic acid, azelaic acid, sebacic acid, undecandionic acid, dodecandionic acid, orthophthalic acid, isophthalic acid, terephthalic acid, maleic acid, fumaric acid, glutaconic acid, traumatic acid and/or muconic acid), an alkali metal salt (e.g., a mono- or di-alkali metal salt) of a C₃ to C₄₀ tricarboxylic acid, for example, citric acid, isocitric acid, aconitic acid, propane-1,2,3-tricarboxylic acid and/or trimesic acid, an alkali metal salt (e.g., a mono-, di-, or tri-alkali metal salt) of a C₄ to C₄₀ tetracarboxylic acid, for example, (2R,3R,11R,12R)-1,4,7,10,13,16-hexaoxacyclooctadecane-2,3,11,12-tetracarboxylic acid, cyclohexane-1,2,4,5-tetracarboxylic acid, 1,2,4,5-benzene-tetracarboxylic acid, and tetrahydrofuran-2,3,4,5-tetracarboxylic acid, an alkali metal salt (e.g., a mono-, di-, tri-, or tetra-alkali metal salt) of a pentacarboxylic acid, for example, benzene-1,2,3,4,5-pentacarboxylic acid, an alkali metal salt (e.g., a mono-, di-, tri-, tetra-, or penta-alkali metal salt) of a hexacarboxylic acid, an alkali metal salt of a C₄ to C₄₀ phosphonotricarboxylic acid, for example, 2-phosphonobutane-1,2,4-tricarboxylic acid, or a combination thereof.

Conditions for the reaction between the polyamine and the organic acid salt or the acid salt are not particularly limited, and may be selected appropriately in light of the types of the polyamine and the types of the organic acid salt or the acid salt. In a non-limiting example, ethylene diamine and disodium citrate may be reacted in an aqueous solvent, for example, water, at a temperature of greater than or equal to about 0° C. to produce an alkylammonium salt compound having the following structure.

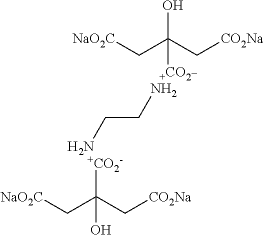

In non-limiting examples, the reaction product of the polyethylene imine and an organic acid salt may be obtained by conducting a reaction with the polyethylene imine and the organic acid salt in a reaction solvent, for example, water, at a temperature of greater than or equal to about 0° C. In other non-limiting examples, the reaction product of the polyethylene imine and an acid salt (e.g., an inorganic acid salt, for example, sodium bisulfate, monosodium phosphate, and disodium phosphate) may be obtained by conducting a reaction with the polyethylene imine and the acid salt in a reaction solvent, for example, water, at a temperature of greater than or equal to about 0° C. The duration for the reaction may be adjusted appropriately and it may be greater than or equal to about 5 minutes, but is not limited thereto.

As such, it is possible to prepare an alkylammonium salt compound having a wide range of molecular weight from a relatively low molecular weight of about 300 or less to a relatively high molecular weight of about several millions and having various structures by selecting the types of the polyamine and the types of the organic acid salt or the acid salt appropriately. In addition, the alkyl ammonium salt compound thus prepared has such a relatively high level of diffusion ability that the alkyl ammonium salt compound may more easily reach the dense layer of the semipermeable membrane to accomplish relatively high water flux at a given osmotic pressure. For example, the alkyl ammonium salt compound may generate a water flux higher than a draw solute including a polyionic salt, for example, MgSO₄. In addition, as stated above, as the alkyl ammonium salt compound may hardly diffuse across the dense layer of the semipermeable membrane toward the feed solution, it becomes possible to keep the reverse solute flux at a relatively low level. Moreover, when a substance having a relatively low level of biotoxicity (e.g., a biocompatible substance) is selected as a raw material (e.g., the polyamine and the organic acid), the reaction product therebetween may be biodegradable and biocompatible (e.g., may have relatively low biotoxicity). Therefore, the draw solute including such an alkyl ammonium salt compound has a reduced level of toxicity and thereby is suitable for a forward osmotic process producing drinking water or living water.

In example embodiments, the alkyl ammonium compound may have a molecular weight of greater than or equal to about 250 g/mol, for example, greater than or equal to about 300 g/mol. For example, the alkyl ammonium compound may have a number average molecular weight of greater than or equal to about 2000 g/mol.

The aforementioned range of the molecular weight makes it possible to accomplish even higher water flux and lower reverse solute flux. Without wishing to be bound by any theory, the aforementioned alkyl ammonium salt compound includes both of the polyamine and/or the organic acid salt (or the acid salt) that are linked with each other via an ionic bond, and thus its molecular chain may be more flexible than a compound having only a covalent bond. Therefore, even when the alkyl ammonium salt compound has a relatively high molecular weight, the alkyl ammonium salt compound may more easily pass through the porous layer of the semipermeable membrane, thereby showing higher water flux than that of the relatively low molecular weight polyionic salt, for example, MgSO₄ and/or MgCl₂. In addition, as the alkyl ammonium salt compound has a relatively high level of molecular weight in comparison with the relatively low molecular weight polyionic salt, the alkyl ammonium salt compound may hardly pass the dense layer of the semipermeable membrane. Therefore, a draw solute including the aforementioned alkyl ammonium salt compound may keep the reverse solute flux at a relatively low level.

As mentioned above, the alkyl ammonium salt compound has a plurality of ionic moieties and relatively high diffusability even when the alkyl ammonium salt compound has a relatively high molecular weight. Therefore, a draw solute including the same may show water flux of greater than or equal to about 10 LMH at an osmotic pressure of about 60 atm. In addition, the alkyl ammonium salt compound may hardly pass across the dense layer of the semipermeable membrane so that the alkyl ammonium salt compound may show a reduced level of reverse solute flux at a given osmotic pressure. For example, a draw solute including the alkyl ammonium salt compound may show a reverse solute flux of less than or equal to about 2 GMH at an osmotic pressure of about 60 atm.

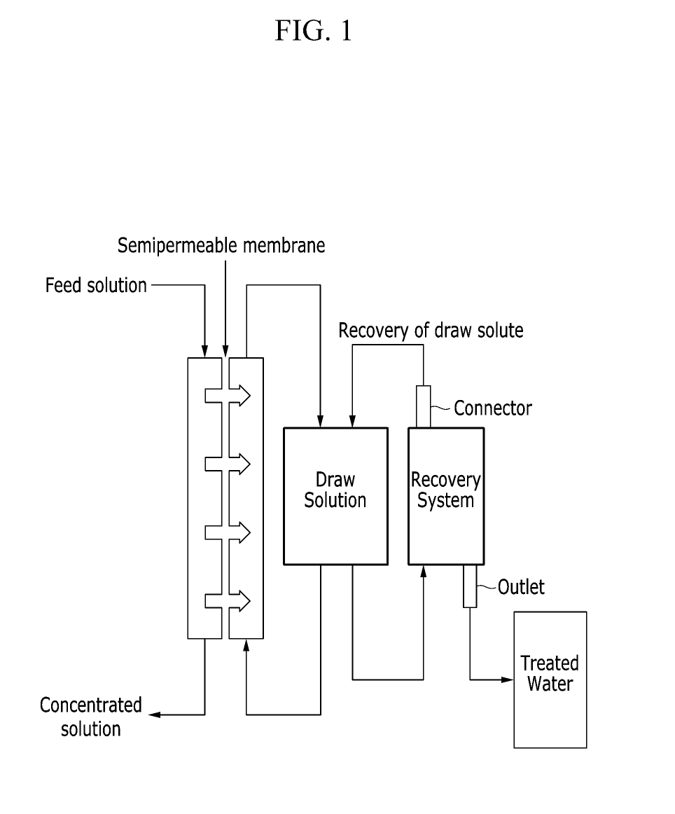

According to example embodiments, a forward osmosis water treatment device may include a feed solution including water and materials to be separated being dissolved in water; an osmosis draw solution including a draw solute including the alkyl ammonium salt compound; a semi-permeable membrane contacting the feed solution on one side and the osmosis draw solution on the other side; a recovery system for removing at least a portion of the draw solute from a treated solution including water that moves from the feed solution to the osmosis draw solution through the semipermeable membrane by osmotic pressure; and a connector for reintroducing the draw solute removed from the recovery system to the osmosis draw solution. FIG. 1 shows a schematic view of a forward osmosis water treatment device according to example embodiments that may be operated by the forward osmosis water treatment method that will be explained hereinafter.

The forward osmosis water treatment device may further include an outlet for discharging treated water produced by removing the draw solute from the treated solution in the recovery system. Types of the outlet are not particularly limited.

The recovery system may include a microfiltration (MF) membrane, an ultrafiltration (UF) membrane, a nanofiltration (NF) membrane, a centrifuge for filtration of the draw solute, a membrane distillation, or a combination thereof. The draw solute thus recovered may be reintroduced to the draw solution via a connector. The alkyl ammonium salt compound may accomplish relatively high osmotic pressure and relatively low reverse solute flux even when the alkyl ammonium salt compound has a relatively high molecular weight, and the alkyl ammonium salt compound may also be recovered by using a (e.g., loose) nanofiltration membrane, making it possible to reduce a recovery cost.

The semi-permeable membrane is permeable to water and impermeable to the materials to be separated. The types of the feed solution are not particularly limited as long as they may be treated in the forward osmosis manner. The materials to be separated may be impurities. Specific examples of the feed solution may include, but are not limited to, sea water, brackish water, ground water and/or waste water. By way of a non-limiting example, the forward osmosis water treatment device may treat sea water to produce drinking water.

Details for the alkyl ammonium salt compound may be the same as those set forth above. The concentration of the osmosis draw solution may be controlled to generate higher osmotic pressure than that of the feed solution.

In example embodiments, a forward osmosis method for water treatment may include contacting a feed solution including water and materials to be separated being dissolved in water and an osmosis draw solution including the aforementioned alkyl ammonium salt compound and water with a semi-permeable membrane positioned therebetween to obtain a treated solution including water that moves from the feed solution to the draw solution through the semi-permeable membrane by osmotic pressure, removing the alkyl ammonium salt compound from the treated solution to obtain treated water, and discharging the treated water.

When the feed solution and the draw solution are brought into contact with the semipermeable membrane disposed therebetween, water is driven to move from the feed solution through the semi-permeable membrane into the osmosis draw solution by osmotic pressure.

Details for the alkyl ammonium salt compound, the semi-permeable membrane, and the forward osmosis process are the same as set forth above.

The following examples illustrate one or more example embodiments in detail. However, they are examples, and this disclosure is not limited thereto.

### EXAMPLE

### [Synthesis of Alkyl Ammonium Salt Compounds]

### Synthesis Example 1

### Reaction Product of Ethylene Diamine and Disodium Citrate

4.51 g of ethylene diamine (purchased from Kanto Chemical Co. Ltd., molecular weight: 60.108) and 39.47 g of disodium citrate sesquihydrate are reacted in 150 mL of water (solvent) at a temperature of 20° C. for 60 minutes, and then the reaction product is precipitated in methanol as a solid. The solid product thus obtained is filtered and dried in a vacuum oven.

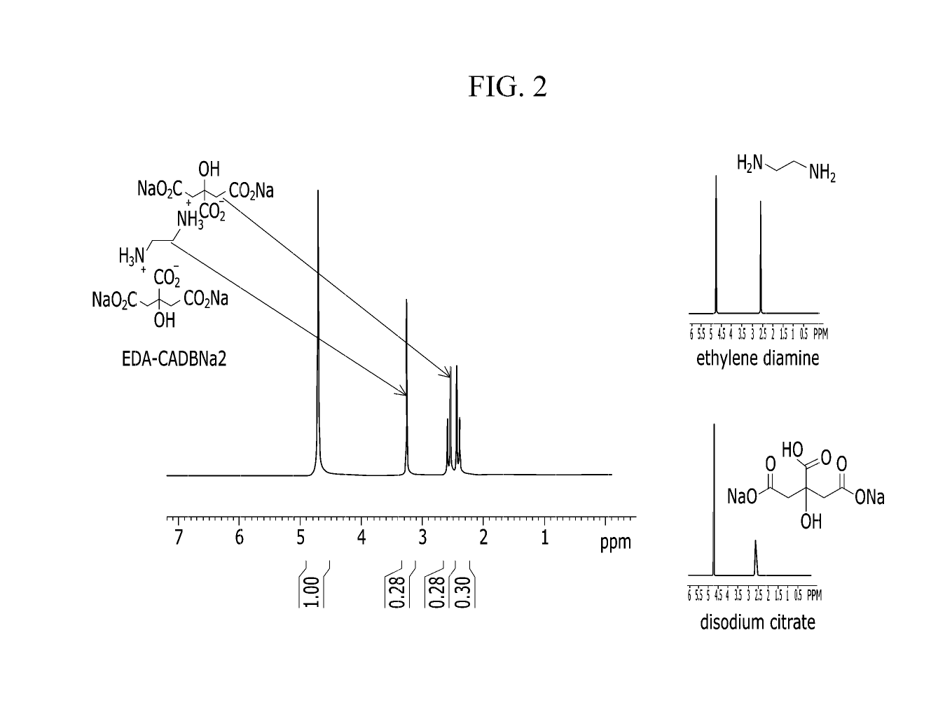

An NMR analysis is made for the obtained alkyl ammonium salt compound being dissolved D₂O by using 300 MHz Bruker NMR equipment. The NMR spectrum of the compound is shown in FIG. 2: ¹H NMR (300 MHz, D₂O), δ (ppm) 3.25 (s, 4H), 2.58 (d, 4H, J=12 Hz), 2.409, (d, 4H J=12 Hz).

From the results of FIG. 2, the reaction product of disodium citrate and ethylene diamine may show a down-shift at a CH₂ peak.

The molecular weight of the alkyl ammonium salt compound is calculated and compiled in Table 1.

### Synthesis Example 2

### Reaction Product of Tetraethylene Pentamine and Disodium Citrate

7.572 g of tetraethylene pentamine (purchased from Kanto Chemical Co. Ltd., molecular weight: 189.30) and 52.622 g of disodium citrate sesquihydrate are reacted in water (solvent) at a temperature of 20° C. for 24 hours, and then the reaction product is precipitated in methanol as a solid. The solid product thus obtained is filtered and dried in a vacuum oven.

An NMR analysis is made for the obtained alkyl ammonium salt compound being dissolved D₂O by using 300 MHz Bruker NMR equipment. The NMR data are as follows: ¹H NMR (300 MHz, D₂O), δ (ppm) 2.65˜3.40 (m, 16H), 2.58 (d, 10H, J=12 Hz), 2.45 (d, 10H, J=12 Hz).

The molecular weight of the alkyl ammonium salt compound is calculated and compiled in Table 1.

### Synthesis Example 3

### Reaction Product of Polyethylene Imine and Disodium Citrate

6.45 g of polyethylene imine (purchased from Aldrich Co. Ltd., molecular weight: Mn=600, Mw=800) and 39.47 g of disodium citrate sesquihydrate are reacted in water (solvent) at a temperature of 20° C. for 24 hours, and then the reaction product is precipitated in methanol as a solid. The solid product thus obtained is filtered and dried in a vacuum oven.

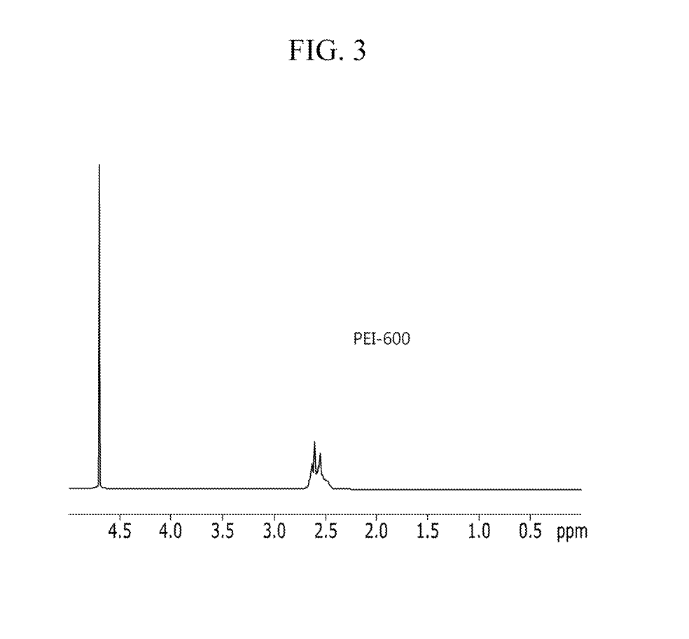

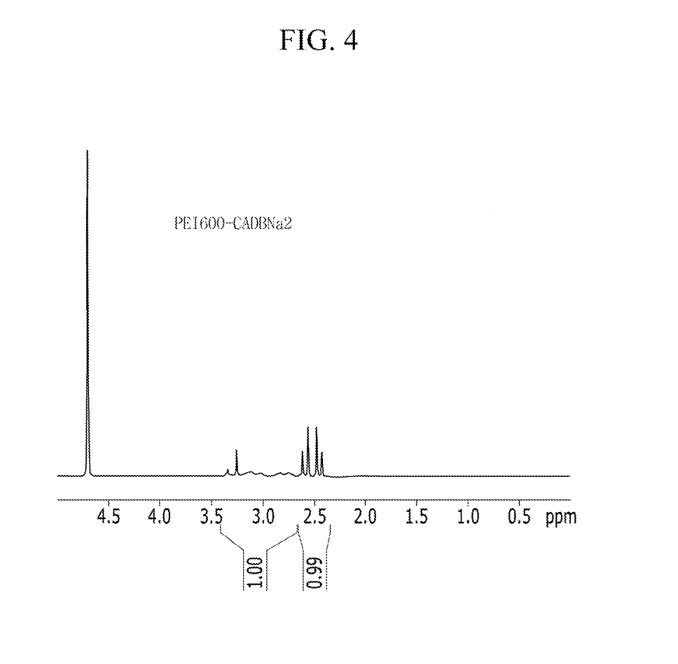

An NMR analysis is made for the obtained alkyl ammonium salt compound being dissolved D₂O by using 300 MHz Bruker NMR equipment. The NMR data for the polyethylene imine as used are shown in FIG. 3 and the NMR data for the compound thus prepared are shown in FIG. 4: ¹H NMR (300 MHz, D₂O), δ(ppm) 2.65˜3.40 (m, 4H), 2.58 (d, 2H, J=12 Hz), 2.45 (d, 2H, J=12 Hz).

### Synthesis Example 4

### Reaction Product of Bishexamethylene Triamine and Disodium Citrate

10.769 g of bishexamethylene triamine (purchased from Aldrich Co. Ltd., Molecular weight: 215.38) and 39.47 g of disodium citrate sesquihydrate are reacted in water (solvent) at a temperature of 20° C. for 24 hours, and then the reaction product is precipitated in methanol as a solid. The solid product thus obtained is filtered and dried in a vacuum oven. An NMR analysis is made for the obtained alkyl ammonium salt compound being dissolved D₂O by using 300 MHz Bruker NMR equipment. The NMR data are as follows: ¹H NMR (300 MHz, D₂O), δ (ppm) 2.91 (m, 8H), 2.58 (d, 6H, J=12 Hz), 2.43 (d, 6H, J=12 Hz), 1.60 (m, 8H), 1.32 (m, 8H).

### Synthesis Example 5

### Reaction Product of Polyethylene Imine and Disodium Citrate

17.2 g of polyethylene imine (purchased from Aldrich Co. Ltd., Mn=1200, 50 wt % in H₂O) and 52.622 g of disodium citrate sesquihydrate are reacted in water (solvent) at a temperature of 20° C. for 24 hours, and then the reaction product is precipitated in methanol as a solid. The solid product thus obtained is filtered and dried in a vacuum oven.

The NMR data for the product are as follows: ¹H NMR (300 MHz, D₂O), δ (ppm) 2.65˜3.40 (m, 4H), 2.58 (d, 2H, J=12 Hz), 2.45 (d, 2H, J=12 Hz).

### Synthesis Example 6

### Reaction Product of Polyethylene Imine and Disodium Citrate

17.2 g of polyethylene imine (purchased from Aldrich Co. Ltd., Mn=1800, 50 wt % in H₂O) and 52.622 g of disodium citrate sesquihydrate are reacted in water (solvent) at a temperature of 20° C. for 24 hours, and then the reaction product is precipitated in methanol as a solid. The solid product thus obtained is filtered and dried in a vacuum oven.

The NMR data for the product are as follows: ¹H NMR (300 MHz, D₂O), δ (ppm) 2.65˜3.40 (m, 4H), 2.58 (d, 2H, J=12 Hz), 2.45 (d, 2H, J=12 Hz).

### Synthesis Example 7

### Reaction Product of Diethylene Triamine and Sodium Bisulfate

10.317 g of diethylene triamine (purchased from Aldrich Co. Ltd., molecular weight: 103.17) and 36.018 g of sodium bisulfate are reacted in water (solvent) at a temperature of 20° C. for 1.5 hours, and then the reaction product is precipitated in methanol as a solid. The solid product thus obtained is filtered and dried in a vacuum oven.

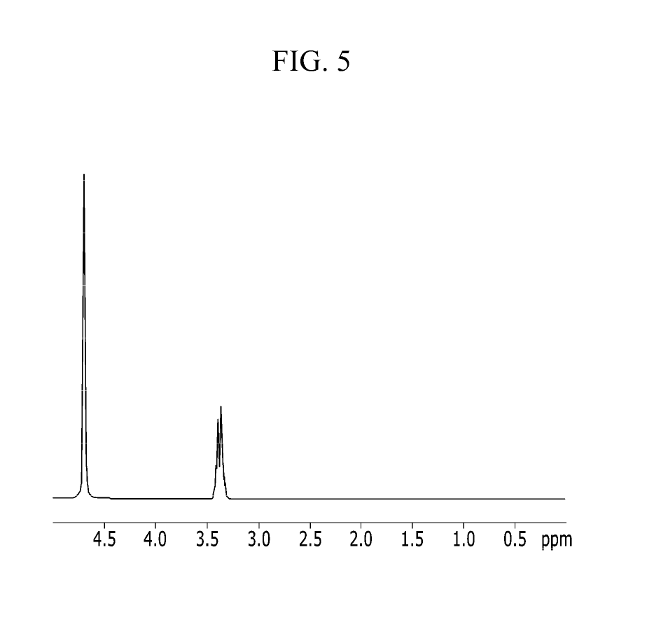

The NMR data for the product are shown in FIG. 5 and compiled as below: ¹H NMR (300 MHz, D₂O), δ (ppm) 3.30˜3.45 (m).

### Synthesis Example 8

### Reaction Product of Polyethylene Imine and Sodium Bisulfate

12.9 g of polyethylene imine (purchased from Aldrich Co. Ltd., molecular weight: Mn=600) and 36.01 g of sodium bisulfate are reacted in water (solvent) at a temperature of 20° C. for 24 hours, and then the reaction product is precipitated in methanol as a solid. The solid product thus obtained is filtered and dried in a vacuum oven. An NMR analysis is made for the obtained alkyl ammonium salt compound being dissolved D₂O by using 300 MHz Bruker NMR equipment.

The NMR data for the product are compiled as below: ¹H NMR (300 MHz, D₂O), δ (ppm) 2.70˜3.70 (m).

### Examples 1 to 8 and Comparative Examples 1 to 3

### Preparation of Draw Solutions

As shown in Table 1, draw solutions of Examples 1 to 8 and Comparative Examples 1 to 3 are prepared using water and each of the alkyl ammonium salt compounds prepared in synthesis examples and MgSO₄ and MgCl₂ as a polyionic salt compound.

TABLE 1

| | | | | The |
|---|---|---|---|---|
| | | | | Number of |
| | | | | acid salts |
| | | | | in one |
| | | | | molecule |
| | | | Types of | (Molecular |
| | Product | Polyamine | (organic) | weight (as |
| | name | type | acid salt | calculated)) |
| Exam- | EDA-CADB- | Ethylene | Disodium | 2 (532 g/mol) |
| ple 1 | Na2 | diamine | citrate | |
| Exam- | TEPA-CADB- | Tetraethylene | | 5 (1370 g/mol) |
| ple 2 | Na2 | pentamine | | |
| Exam- | PEI600- | Polyethylene | | ~14 |
| ple 3 | CADB-Na2 | imine | | (3906 g/mol) |
| | | (Mn 600) | | |
| Exam- | BHMTA- | Bishexamethylene | | 3 (923 g/mol) |
| ple 4 | CADB- | triamine | | |
| | Na2 | | | |
| Exam- | PEI600- | Polyethylene | | ~12.6 |
| ple 5 | 0.9CADB | imine | | (3576 g/mol) |
| | Na2 | (Mn 600) | | |
| Exam- | PEI1800- | Polyethylene | | ~42 |
| ple 6 | CADB- | imine | | (11,718 g/mol) |
| | Na2 | (Mn 1800) | | |
| Exam- | DETA-SA- | Diethylene | Sodium | 3 (463 g/mol) |
| ple 7 | Na | triamine | bisulfate | |
| Exam- | PEI600- | Polyethylene | (SA), | ~14 |
| ple 8 | SA-Na | imine | | (2338 g/mol) |
| | | (Mn 600) | | |
| Comp. | MgCl₂ | — | Polyionic | ~3* (the |
| Ex 1 | | | salt | number of |
| | | | | ions) |
| Comp. | MgSO₄ | — | Polyionic | ~2* (the |
| Ex 2 | | | salt | number of |
| | | | | ions) |

### Experimental Example 1

### Evaluation of Osmotic Pressure I

The draw solutions including the alkyl ammonium salt compound of Examples 1 to 8 and the draw solutions including the compound of Comparative Examples 1 and 2 are prepared to have various concentrations. Osmotic pressure of each draw solution is measured by using osmotic pressure measurement equipment (Osmomat 010) in accordance with the freezing point lowering method. Results are shown in Table 2.

### Experimental Example 2

### Water Flux and Reverse Solute Flux

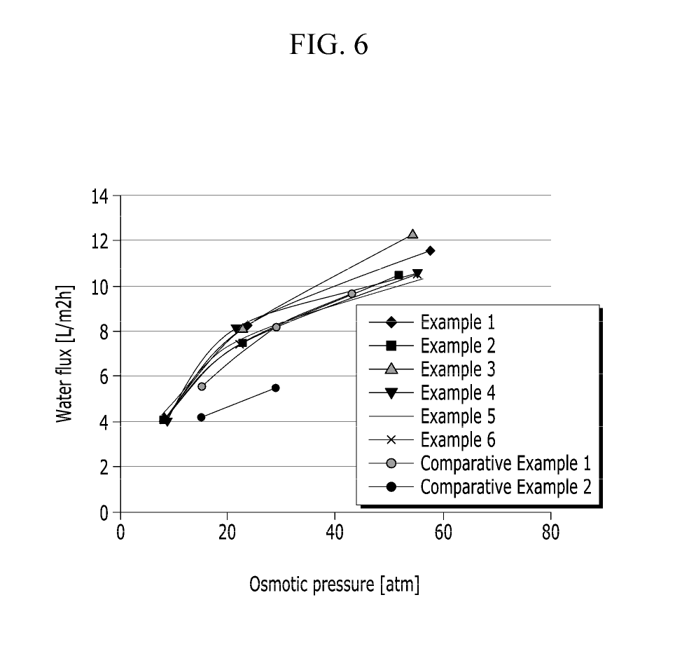

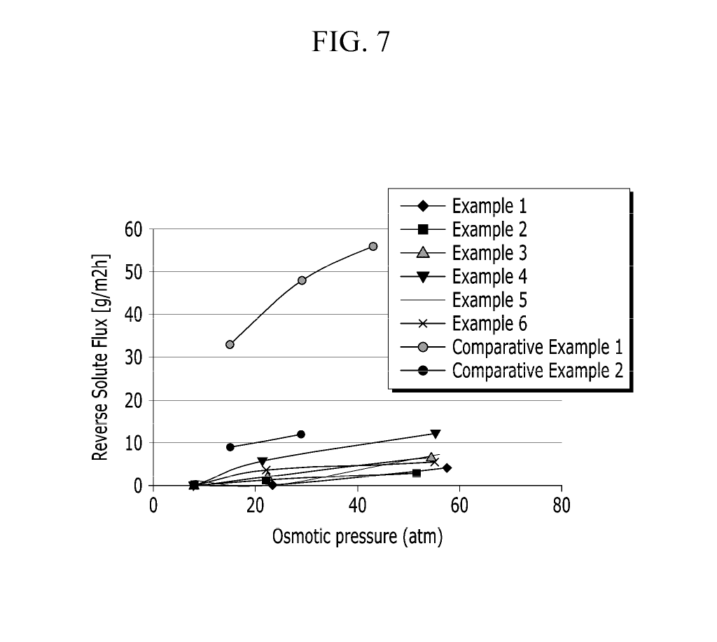

With respect to each of the draw solutions of Examples 1 to 6 and Comparative Examples 1 and 2, an osmotic flow analysis is conducted in accordance with the following manner. The osmotic flow is evaluated with a homemade, U-shaped semi-dynamic forward osmosis apparatus. To test performance of the draw solute, a semi-permeable commercialized FO membrane (cellulose trifluoroacetate) (Hydration Technology Innovation (HTI), USA) is placed in the middle of the apparatus. Each side is filled with distilled water as a feed solution and a draw solution with predetermined or given concentrations, respectively. The selective layer is faced toward the feed solutions and osmotic water flux from feed to draw solutions is calculated from the volumetric change of each solution during one hour after 30 min. The reversed solute flux from draw to feed solution through the membrane is measured by conductivity, inductively coupled plasma optical emission spectroscopy (ICP-OES), and total organic carbon (TOC). The results are shown in FIG. 6, FIG. 7, and Table 3.

TABLE 2

| | Concentration | |
|---|---|---|
| | (mg/ml) | Osmotic pressure (atm) |
| Example 1 | 29.6 | 5.8 |
| EDA-CADB-Na2 | 118.4 | 21.8 |
| | 177.6 | 33.8 |
| | 236.8 | 50.4 |
| | 266.4 | 61.1 |
| Example 2 | 34.2 | 6.3 |
| TEPA-CADB-Na2 | 68.5 | 11.8 |
| | 102.7 | 17.5 |
| | 205.43 | 37.5 |
| | 301.2 | 67.1 |
| Example 3 | 78.1 | 12.0 |
| PEI600-CADB-Na2 | 156.2 | 25.3 |
| | 234.4 | 42.9 |
| | 273.4 | 54.4 |
| | 292.9 | 67.1 |
| Example 4 | 46.2 | 8.6 |
| BHMTA-CADB-Na2 | 92.3 | 17.2 |
| | 184.6 | 38.2 |
| | 249.2 | 59.3 |
| Example 5 | 45.2 | 7.8 |
| PEI1200-CADB-Na2 | 138.3 | 22.6 |
| | 278.2 | 55.7 |
| Example 6 | 46.6 | 7.9 |
| PEI1800-CADB-Na2 | 140.0 | 22.2 |
| | 280.0 | 55.2 |
| Example 7 | 46.3 | 10.2 |
| DETA-SA-Na | 92.6 | 18.2 |
| | 185.2 | 20.4 |
| | 277.8 | 26.8 |
| | 370.4 | 37.6 |
| | 463.0 | 62.8 |
| Example 8 | 36.3 | 7.8 |
| PEI600-SA-Na | 72.5 | 14.1 |
| | 145.0 | 26.7 |
| | 290.0 | 27.7 |
| | 435.0 | 35.3 |
| Comp. Example 1 | 19.0 | 11.9 |
| MgCl₂ | 38.1 | 25.2 |
| | 76.2 | 60.6 |
| Comp. Example 2 | 60.2 | 11.7 |
| MgSO₄ | 120.4 | 25.8 |
| | 240.7 | 62.4 |

TABLE 3

| | | Water | Reverse |
|---|---|---|---|
| | Osmotic | flux | solute flux |
| | pressure | (LMH) | (GMH) |
| EDA-CADB-Na2 | 8.3 | 4.06 | 0 |
| (Example 1) | 23.5 | 8.25 | 0.02 |
| | 57.6 | 11.58 | 0.41 |
| TEPA-CADB-Na2 | 8.1 | 4.12 | 0 |
| (Example 2) | 22.2 | 7.45 | 0.14 |
| | 51.7 | 10.46 | 0.29 |
| PEI600-CADB-Na2 | 8.3 | 4.15 | 0 |
| (Example 3) | 22.6 | 8.04 | 0.22 |
| | 54.5 | 12.32 | 0.68 |
| BHMTA-CADB-Na2 | 8.1 | 3.86 | 0 |
| (Example 4) | 21.5 | 8.1 | 0.59 |
| | 55.3 | 10.59 | 1.22 |
| PEI1200-CADB-Na2 | 7.8 | 4.34 | 0.14 |
| (Example 5) | 22.6 | 7.69 | 0 |
| | 55.7 | 10.29 | 0.73 |
| PEI1800-CADB-Na2 | 7.89 | 4.03 | 0 |
| (Example 6) | 22.24 | 7.42 | 0.37 |
| | 55.23 | 10.51 | 0.55 |
| MgCl₂ | 15.0 | 5.6 | 3.3 |
| (Comp. Example 1) | 29.0 | 8.2 | 4.8 |
| | 43.0 | 9.7 | 5.6 |
| MgSO₄ | 15.0 | 4.2 | 0.9 |
| (Comp. Example 2) | 29.0 | 5.5 | 1.2 |

The results of FIG. 6 and FIG. 7 confirm that the draw solutions of Examples 1 to 6 may exhibit a higher level of water flux than the draw solutions of Comparative Examples at the same level of osmotic pressure.

The results of Table 3 confirm that the draw solutions of Examples 1 to 6 may show a relatively low level of reverse solute flux together with relatively high water flux.

While example embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims.

What is claimed is:

1. A draw solute comprising a water-soluble alkyl ammonium salt compound, the water-soluble alkyl ammonium salt compound including,
an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation.

2. The draw solute of claim 1, wherein the ionic moiety includes one of —COOM, —SO₃M, —OSO₃M, —OPO₃M₂, —OPO₃MH, —PO₃M₂, —OPO₃Me, and a combination thereof, wherein M is one of Li, Na, K and Rb, and Me is one of Ca, Mg, Sr and Ba.

3. The draw solute of claim 1, wherein the at least two ammonium cationic moieties includes one of a primary ammonium cation, a secondary ammonium cation, a tertiary ammonium cation and a combination thereof.

4. The draw solute of claim 1, wherein the water-soluble alkyl ammonium salt compound includes:
a reaction product of a polyamine compound having at least two amine groups with one of an organic acid salt having the ionic moiety together with a functional group capable of donating hydrogen to one of the at least two amine groups and an acid salt having the ionic moiety.

5. The draw solute of claim 4, wherein
the at least two amine groups are the same as or different from each other and are independently selected from one of a primary amine, a secondary amine, and a tertiary amine, and
the functional group capable of donating hydrogen to one of the at least two amine groups is one of a carboxyl group (—COOH), a sulfonic acid group (—SO₃H), a phosphonic acid group (—PO₃H₂), a phosphoric acid group (—OPO₃H₂), and a combination thereof.

6. The draw solute of claim 4, wherein the polyamine compound is a compound represented by Chemical Formula 1: 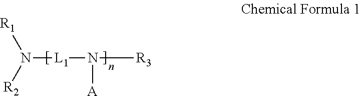
wherein
R₁, R₂, and R₃ are the same or different, and are each independently one of a hydrogen and a substituted or unsubstituted C₁ to C₃₀ monovalent aliphatic hydrocarbon group,
L₁ is a substituted or unsubstituted C₁ to C₃₀ divalent aliphatic hydrocarbon group,
A is one of a hydrogen, a substituted or unsubstituted C₁ to C₃₀ monovalent aliphatic hydrocarbon group, a substituted or unsubstituted aminoalkyl group, and a moiety represented by Chemical Formula 2, and
n is an integer of greater than or equal to 1, and when n is at least 2, each L₁ is the same or different and each A is the same or different: 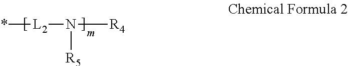
wherein
L₂ is a substituted or unsubstituted C₁ to C₃₀ divalent aliphatic hydrocarbon group,
R₄ and R₅ are the same or different and are each independently one of a hydrogen, a substituted or unsubstituted C1 to C30 monovalent aliphatic hydrocarbon group, and a moiety represented by Chemical Formula 2,
m is an integer greater than or equal to 1, and
* is a portion that is linked to the nitrogen atom.

7. The draw solute of claim 4, wherein the polyamine compound includes one of polyethylene imine, polypropylene imine, bishexamethylenetriamine, ethylenediamine, 1,2-diamino propane, 1,3-diamino propane, N-methylene diamine(N-methylethylenediamine), 1,4-diamino butane, 3-(methylamino)propylamine, N,N′-dimethylethylenediamine, N,N-dimethyl ethylenediamine, N-ethylethylenediamine, N-methyl-1,3-diamino propane, 1-dimethyl amino2-propylamine, 3-(dimethyl amino)-1-propylamine, Cadaverine, N,N′-dimethyl-1,3-propanediamine, N,N,N′,N′-tetramethyl diaminomethane, N,N,N′-trimethyl ethylenediamine, N-isopropyl ethylenediamine, N-propylethylenediamine, 2-(aminomethyl)-2-methyl-1,3-propanediamine, 2-(aminomethyl)-2-methyl-1,3-propanediamine trichloride, N-(2-aminoethyl)-1,3-propanediamine, 1,5-diamino-2-methyl pentane, N,N′-diethylethylenediamine, N,N,N′,N′-tetramethyl ethylenediamine, N,N-diethylethylenediamine, N,N-dimethyl-N′-ethylethylenediamine, N-butylethylenediamine, N-isopropyl-1,3-propanediamine, N-propyl-1,3-propanediamine, bis(3-aminopropyl)amine, triethylenetetramine, 1,3-bis(ethylamino)propane, 1,7-diaminoheptane, 3-(diethylamino)propylamine, N,N′-diethyl-1,3-propanediamine, N,N,2,2-tetramethyl-1,3-propanediamine, N,N,N′,N′-tetramethyl-1,3-propanediamine, N,N-diethyl-1,3-propanediamine, N,N-diethyl-N′-methyl ethylenediamine, 3,3′-diamino-N-methyl dipropylamine, N′-isopropyl diethylenetriamine(N′-Isopropyldiethylenetriamine), tris(dimethyl amino)methane, N,N′-bis(2-aminoethyl)-1,3-propanediamine, trans-N,N,N′,N′-tetramethyl-2-butene-1,4-diamine, 1,8-diaminooctane, N,N′-dimethyl-1,6-hexane diamine, N,N,N′,N′-tetramethyl-1,3-butanediamine, N,N,N′-triethylethylenediamine, N-hexyl ethylenediamine, bis[2-(N,N-dimethyl amino)ethyl]ether, N,N-diethyldiethylenetriamine, N,N-dimethyl dipropylene triimine, 1,2-bis(3-aminopropylamino)ethane, tetraethylene pentamine, 2,4,6-trimethyl-m-phenylenediamine, N-benzylethylenediamine, and a combination thereof.

8. The draw solute of claim 4, wherein
the organic acid salt includes an alkali metal salt of a polycarboxylic acid, and
the acid salt includes one of a sodium bisulfate, monosodium phosphate, disodium phosphate, and a combination thereof.

9. The draw solute of claim 8, wherein the alkali metal salt of a polycarboxylic acid includes one of an alkali metal salt of a C₂ to C₄₀ dicarboxylic acid, an alkali metal salt of a C₃ to C₄₀ tricarboxylic acid, an alkali metal salt of a C₄ to C₄₀ tetracarboxylic acid, an alkali metal salt of a C₅ to C₄₀ pentacarboxylic acid, an alkali metal salt of a C₆ to C₄₀ hexacarboxylic acid, an alkali metal salt of a C₄ to C₄₀ phosphonotricarboxylic acid, and a combination thereof.

10. The draw solute of claim 1, wherein the water-soluble alkyl ammonium compound has a molecular weight of greater than or equal to about 200 g/mol.

11. The draw solute of claim 1, wherein the water-soluble alkyl ammonium compound includes a polymeric compound having a number average molecular weight of greater than or equal to about 2,000 g/mol.

12. The draw solute of claim 1, wherein the draw solute has a water flux of greater than or equal to about 10 LMH and a reverse solute flux of less than or equal to about 2 GMH at an osmotic pressure of about 60 atm.

13. A forward osmosis water treatment device comprising:
a feed solution including water and materials dissolved in the water;
an osmosis draw solution including water and a draw solute, the draw solute including a water-soluble alkyl ammonium salt compound, the water-soluble alkyl ammonium salt compound including an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation;
a semi-permeable membrane having a first side and an opposing second side, the semi-permeable membrane having the feed solution contacting the first side and the osmotic draw solution contacting the second side;
a recovery system configured to remove at least a portion of the draw solute from a treated solution including the water from the feed solution that moves to the osmotic draw solution through the semi-permeable membrane by osmotic pressure; and
a connector configured to reintroduce the draw solute removed by the recovery system back into the osmotic draw solution contacting the semi-permeable membrane.

14. The forward osmosis water treatment device of claim 13, wherein the water-soluble alkyl ammonium salt compound includes:
a reaction product between a polyamine compound having at least two amine groups, and
one of an organic acid salt having the ionic moiety together with a functional group capable of donating hydrogen to one of the at least two amine groups and an acid salt having the ionic moiety.

15. The forward osmosis water treatment device of claim 13, further comprising:
an outlet configured to discharge treated water produced by removing the draw solute from the treated solution in the recovery system.

16. The forward osmosis water treatment device of claim 13, wherein the recovery system includes one of a microfiltration (MF) membrane, an ultrafiltration (UF) membrane, a loose nanofiltration (NF) membrane, a centrifugal separator, and a membrane distillation.

17. A forward osmosis method for water treatment, the method comprising:
contacting a feed solution, the feed solution including water and materials dissolved in the water, and an osmotic draw solution including a draw solute with a semi-permeable membrane therebetween to obtain a treated solution including the water that moves from the feed solution to the osmotic draw solution through the semi-permeable membrane by osmotic pressure, the draw solute including a water-soluble alkyl ammonium salt compound, the water-soluble alkyl ammonium salt compound including an ionic moiety and at least two ammonium cationic moieties, the ionic moiety including an anion selected from a carbonate anion (COO⁻), a sulfonate anion (SO₃⁻), a sulfate anion (SO₄²⁻), a phosphonate anion (PO₃²⁻), and a phosphate anion (PO₄³⁻), and a cation selected from an alkali metal cation and an alkaline earth metal cation;
removing at least a portion of the draw solute by filtration to obtain treated water; and
discharging the treated water.

18. The forward osmosis method for water treatment of claim 17, wherein the water-soluble alkyl ammonium salt compound includes:
a reaction product between a polyamine compound having at least two amine groups, and
one of an organic acid salt having the ionic moiety together with a functional group capable of donating hydrogen to one of the at least two amine groups and an acid salt having the ionic moiety.
